(19) United States
(12) Patent Application Publication
(10) Pub. No.: US 2020/0147209 A1
(43) Pub. Date: May 14, 2020

(54) ALUM-CONTAINING COATING FORMULATIONS FOR MICRONEEDLE VACCINE PATCHES

(71) Applicant: 3M INNOVATIVE PROPERTIES COMPANY, St. Paul, MN (US)

(72) Inventor: Peter R. Johnson, Eagan, MN (US)

(21) Appl. No.: 16/744,247

(22) Filed: Jan. 16, 2020

Related U.S. Application Data
(63) Continuation of application No. 15/544,993, filed on Jul. 20, 2017.
(60) Provisional application No. 62/108,155, filed on Jan. 27, 2015.

(51) Int. Cl. A61K 39/39; A61L 31/08; A61L 31/10; A61L 31/16; A61K 39/00; A61K 47/38; A61K 47/26; A61K 47/02; A61K 9/06; A61K 9/00

(52) U.S. Cl. CPC A61K 39/39; A61M 2037/0053; A61L 31/10; A61L 31/16; A61K 39/00; A61K 47/38; A61K 47/26; A61K 47/02; A61K 9/06; A61K 9/0021; Y02A 50/39; Y02A 50/388; Y02A 50/466; Y02A 50/484; Y02A 50/464; A61L 2300/256; A61L 2300/102; A61L 31/088

(57) ABSTRACT

Compositions for coating microneedles with aluminum-adjuvanted vaccines are provided comprising an aluminum-containing wet gel suspension selected from aluminum hydroxide wet gel suspension and aluminum phosphate wet gel suspension; a vaccine in an amount effective to stimulate an immune response in a mammal; a sugar, sugar alcohol, or combinations thereof; and a thickener. Some embodiments of the compositions have a viscosity of 500 to 30,000 cps when measured at 100 s⁻¹ and temperature of 25° C. Microneedle devices coated with the compositions, as well as methods of forming the compositions and coating the microneedles, and methods of maximizing the aluminum content of vaccine-coated microneedle arrays are also provided.

### BACKGROUND

Devices including arrays of relatively small structures, sometimes referred to as microneedles or micro-pins, have been disclosed for use in connection with the delivery of therapeutic agents such as vaccines through the skin and other surfaces. The devices are typically pressed against the skin in an effort to pierce the stratum corneum such that the therapeutic agents and other substances can pass through that layer and into the tissues below.

Microneedle devices having a fluid reservoir and conduits through which a therapeutic substance may be delivered to the skin have been proposed, but microneedle devices having a dried coating on the surface of a microneedle array have desirable features compared to fluid reservoir devices, as they are generally simpler and can directly inject a therapeutic substance into the skin without the need for providing reliable control of fluid flow through very fine channels in the microneedle device.

In the field of immunology it has been well known for many years that immune response to certain antigens which are otherwise weakly immunogenic can be enhanced through the use of vaccine adjuvants. Such adjuvants potentiate the immune response to specific antigens and are therefore the subject of considerable interest and study within the medical community. Alum, or aluminum compounds, are the only adjuvants widely used in vaccines, and in some cases are the only approved vaccine adjuvants.

Therefore, there is a need for compositions and methods for coating microneedles with aluminum adjuvant-containing coating formulations.

### SUMMARY

The present invention provides, compositions and methods for coating microneedles and microneedle arrays with aluminum-adjuvanted vaccines.

In one aspect of the invention, the present disclosure provides a composition comprising: an aluminum-containing wet gel suspension selected from aluminum hydroxide wet gel suspension and aluminum phosphate wet gel suspension; a vaccine in an amount effective to stimulate an immune response in a mammal; a sugar, sugar alcohol, or combinations thereof; and a thickener; wherein the composition has a viscosity of 500 to 30,000 cps when measured at 100 s⁻¹ and temperature of 25° C.

In another aspect, the present disclosure provides a device comprising: a microneedle array comprising a substrate and a plurality of microneedles; and any of the compositions provided herein coated on at least a portion of one or more of the microneedles.

In another aspect, the present disclosure provides a method of forming an aluminum-adjuvanted vaccine formulation comprising: providing a first aluminum-containing wet gel suspension selected from aluminum hydroxide wet gel suspension and aluminum phosphate wet gel suspension; concentrating the aluminum-containing wet gel suspension to produce a second aluminum-containing wet gel suspension; mixing at least one vaccine into the second aluminum-containing wet gel suspension in an amount effective to stimulate an immune response in a mammal to form the aluminum-adjuvanted vaccine formulation.

In another aspect, the present disclosure provides a method for maximizing the aluminum content of a vaccine-coated microneedle array comprising: providing a microneedle array comprising a microneedle substrate and a plurality of microneedles; forming aluminum-adjuvanted vaccine formulation by providing a first aluminum-containing wet gel suspension selected from aluminum hydroxide wet gel suspension and aluminum phosphate wet gel suspension; concentrating the aluminum-containing wet gel suspension to produce a second aluminum-containing wet gel suspension; mixing at least one vaccine into the second aluminum-containing wet gel suspension in an amount effective to stimulate an immune response in a mammal to form the aluminum-adjuvanted vaccine formulation; and bringing at least a portion of the plurality of microneedles into contact with the aluminum-adjuvanted vaccine formulation, thereby transferring at least a portion of the aluminum-adjuvanted vaccine formulation to the microneedle array to form a wet-coated microneedle array.

As used herein, certain terms will be understood to have the meaning set forth below:

“Array” refers to the medical devices described herein that include one or more (in some embodiments, a plurality) structures capable of piercing the stratum corneum to facilitate the transdermal delivery of therapeutic agents or the sampling of fluids through or to the skin.

“Microstructure,” “microneedle” or “microarray” refers to the specific microscopic structures associated with the array that are capable of piercing the stratum corneum to facilitate the transdermal delivery of therapeutic agents or the sampling of fluids through the skin. By way of example, microstructures can include needle or needle-like structures as well as other structures capable of piercing the stratum corneum.

“Aluminum” refers to elemental aluminum. “Aluminum salt” refers to salts of aluminum such as, for example, aluminum hydroxide or aluminum phosphate and is used interchangeably with “alum”.

The features and advantages of the present invention will be understood upon consideration of the detailed description of the preferred embodiment as well as the appended claims. These and other features and advantages of the invention may be described below in connection with various illustrative embodiments of the invention. The above summary of the present invention is not intended to describe each disclosed embodiment or every implementation of the present invention. The Figures and the detailed description which follow more particularly exemplify illustrative embodiments.

### BRIEF DESCRIPTION OF THE DRAWINGS

FIG. 1 is a flow chart illustrating one embodiment of a method of forming an alum-adjuvanted vaccine formulation according to the present invention;

FIG. 2 is a photomicrograph of a portion of a coated microneedle array coated with the composition of Example 3.

### DETAILED DESCRIPTION

In the following description, reference is made to the accompanying figures that form a part hereof, and in which are shown by way of illustration several specific embodiments. It is to be understood that other embodiments are contemplated and may be made without departing from the scope or spirit of the present disclosure. The following detailed description, therefore, is not to be taken in a limiting sense.

All scientific and technical terms used herein have meanings commonly used in the art unless otherwise specified. The definitions provided herein are to facilitate understanding of certain terms used frequently herein and are not meant to limit the scope of the present disclosure.

Unless otherwise indicated, all numbers expressing feature sizes, amounts, and physical properties used in the specification and claims are to be understood as being modified in all instances by the term “about.” Accordingly, unless indicated to the contrary, the numerical parameters set forth in the foregoing specification and attached claims are approximations that can vary depending upon the desired properties sought to be obtained by those skilled in the art utilizing the teachings disclosed herein.

The recitation of numerical ranges by endpoints includes all numbers subsumed within that range (e.g., 1 to 5 includes 1, 1.5, 2, 2.75, 3, 3.80, 4, and 5) and any range within that range.

As used in this specification and the appended claims, the singular forms “a”, “an”, and “the” encompass embodiments having plural referents, unless the content clearly dictates otherwise. As used in this specification and the appended claims, the term “or” is generally employed in its sense including “and/or” unless the content clearly dictates otherwise. Delivery of vaccine formulation via a microneedle device is a growing field. Delivery of vaccine formulations often requires or benefits from addition of an adjuvant to enhance the immune response to the specific vaccines. Alum, or aluminum compounds, are the only adjuvants widely used in vaccines, and in some cases are the only approved vaccine adjuvants. Aluminum compounds, such as the most commonly used aluminum hydroxide and aluminum phosphate, can present some difficulties for inclusion in coating formulations for coating onto microneedle devices. Microneedle devices are typically coated with aqueous solutions, so insoluble salts such as aluminum hydroxide and aluminum phosphate cannot be used to make such aqueous solution formulations. In addition, because it is desirable to produce uniform coatings for microneedle devices in order to ensure accurate and uniform doses across all or most devices produced, stable, uniform solutions are typically used for coating the microneedle devices. Suspensions that may settle and result in a varying distribution of components, such as those containing insoluble compounds, therefore, present a problem in achieving uniformity of coatings. Additionally, typical injectable vaccine formulations can contain large amounts of alum adjuvants due to the large amount of formulation being injected. Microneedles devices, particularly coated microneedle devices, however, utilize a limited amount of vaccine formulation, and therefore adjuvant, due to their small size. It is important that, in addition to an adequate amount of vaccine, an adequate amount of adjuvant be present in order to enhance the immune response to the vaccine. Thus, the ability to provide a stable, uniform coating composition with maximum aluminum adjuvant content for coating on one or more desired locations on a microneedle array is important for delivering vaccines via microneedle device. It is further desirable to be able to provide an aluminum adjuvanted vaccine coating formulation that can be easily coated onto a microneedle device through methods such as dip coating. It has now been found that stable, uniform compositions and formulations providing maximal aluminum content for enhanced immunogenicity of included vaccines can be achieved for coating microneedles devices. Such compositions, as well as methods for forming and using such compositions, are described in further detail below.

Disclosed herein are compositions that can be utilized to coat microneedle arrays. In some embodiments, the compositions are alum-adjuvanted vaccine formulations. The compositions can be referred to as formulations, coatings, or coating formulations. Devices comprising the compositions, as wells as methods of forming the compositions or formulations, methods for maximizing the alum content of a vaccine-coated microneedle array, and methods for delivering an alum-adjuvanted vaccine to a mammal are also disclosed herein.

Compositions disclosed herein generally include aluminum-containing wet gel suspensions, such as aluminum hydroxide wet gel suspension or aluminum phosphate wet gel suspension. Such suspensions generally comprise water and an insoluble aluminum salt. Exemplary aluminum-containing wet gel suspensions can include aluminum hydroxide wet gel suspension, e.g. ALHYDROGEL® (2% w/w), available from Brenntag Biosector, catalogue number 843261. Other exemplary aluminum-containing wet gel suspensions can include aluminum phosphate wet gel suspensions, e.g. ADJU-PHOS®, available from Brenntag Biosector. In some embodiments, aluminum-containing wet gel suspensions can comprise an aluminum hydroxide wet gel suspension comprising 9 mg/ml to 11 mg/ml aluminum, 9.5 mg/ml to 22 mg/ml aluminum, or 14 mg/ml to 22 mg/ml aluminum. For example, some embodiments may comprise ALHYDROGEL®, which contains 9 mg/ml to 11 mg/ml aluminum. In some embodiments, aluminum-containing wet gel suspensions can comprise concentrated aluminum hydroxide wet gel suspensions. For example, ALHYDROGEL® may be concentrated using methods further described below to achieve concentrations of aluminum ranging from 9.5 mg/ml to 22 mg/ml, or 14 mg/ml to 22 mg/ml for use in the compositions and methods described herein. In some embodiments, the aluminum-containing wet gel suspensions, such as aluminum hydroxide wet gel suspensions, can be diluted to provide aluminum concentrations ranging from 0.10 mg/ml to 10 mg/ml.

In some embodiments, aluminum-containing wet gel suspensions can comprise an aluminum phosphate wet gel suspension comprising 4.5 mg/ml to 5.5 mg/ml aluminum, 5 mg/ml to 15 mg/ml aluminum, 6 mg/ml to 15 mg/ml aluminum, or 7 mg/ml to 10 mg/ml aluminum. For example, some embodiments may comprise ADJU-PHOS®, which contains 4.5 mg/ml to 5.5 mg/ml aluminum. In some embodiments, aluminum-containing wet gel suspensions can comprise concentrated aluminum phosphate wet gel suspensions. For example, ADJU-PHOS® may be concentrated using methods further described below to achieve concentrations of aluminum ranging from 5 mg/ml to 15 mg/ml, 6 mg/ml to 15 mg/ml aluminum, or 7 mg/ml to 10 mg/ml aluminum for use in the compositions and methods described herein.

In some embodiments, the aluminum-containing wet gel suspensions can be concentrated. For example, the aluminum-containing wet gel suspensions can be centrifuged and a portion of the supernatant can be removed, thus increasing the per-volume aluminum content of the suspension. In some embodiments, aluminum-containing wet gel suspensions can be concentrated through evaporation or other known methods of concentration. In some embodiments, aluminum-containing wet gel suspensions can be diluted, such as by addition of water, buffer, or other solvent.

In some embodiments, the aluminum-containing wet gel suspension comprises 0.01 wt.-% to 5 wt.-% aluminum. In some embodiments, the aluminum-containing wet gel suspension comprises 0.1 wt.-% to 2 wt.-% aluminum. In some embodiments, the aluminum-containing wet gel suspension comprises 5 mg/ml to 22 mg/ml aluminum.

The alum, provided as aluminum-containing wet gel suspensions can act as an adjuvant for the vaccines included in the compositions. An adjuvant is an agent that modifies the effect of another agent (in this case the vaccine). Adjuvants are often utilized to enhance the recipient's immune response to the vaccine.

In some embodiments, the water present in the aluminum-containing wet gel suspensions can act as a solvent, such that it may dissolve or disperse any active pharmaceutical ingredient and excipients. In some embodiments, the compositions disclosed herein can also include co-solvents in addition to water. In some embodiments, the compositions can optionally include additional solvents (also referred to as co-solvents) such as ethanol, iospropanol, methanol, propanol, butanol, propylene glycol, dimethysulfoxide, glycerin, 1-methyl-2-pryrrolidinone, or N,N-dimethylformamide.

The compositions disclosed herein generally include at least one vaccine. Examples of suitable vaccines include DNA vaccine, cellular vaccines such as a dendritic cell vaccine, recombinant protein vaccine, therapeutic cancer vaccine, anthrax vaccine, flu vaccine, Lyme disease vaccine, rabies vaccine, measles vaccine, mumps vaccine, chicken pox vaccine, small pox vaccine, hepatitis vaccine, hepatitis A vaccine, hepatitis B vaccine, hepatitis C vaccine, pertussis vaccine, rubella vaccine, diphtheria vaccine, encephalitis vaccine, Japanese encephalitis vaccine, respiratory syncytial virus vaccine, yellow fever vaccine, ebola virus vaccine, polio vaccine, herpes vaccine, human papilloma virus vaccine, rotavirus vaccine, pneumococcal vaccine, meningitis vaccine, whooping cough vaccine, tetanus vaccine, typhoid fever vaccine, cholera vaccine, tuberculosis vaccine, severe acute respiratory syndrome (SARS) vaccine, HSV-1 vaccine, HSV-2 vaccine, HIV vaccine and combinations thereof. The term “vaccine” thus includes antigens in the forms of proteins, peptides, lipoproteins, glycoproteins, polysaccarides, lipopolysaccharides, oligosaccarides, glycolipids, polynucleotide sequences, weakened or killed viruses, virus particles, virus-like particles, weakened or killed bacteria, bacterial cell walls, toxoids, and desensitizing agents such as cat, dust, or pollen allergens. Additional examples of suitable vaccines are described in United States Patent Application Publication Nos. 2004/0049150, 2004/0265354, and US2006/0195067, the disclosures of which are incorporated herein by reference.

In some embodiments, the compositions can include at least one sugar, sugar alcohol, or combinations thereof. Exemplary sugars can include for example non-reducing sugars such as raffinose, stachyose, sucrose, and trehalose; and reducing sugars such as monosaccharides and disaccharides. Exemplary monosacharides can include apiose, arabinose, digitoxose, fucose, fructose, galactose, glucose, gulose, hamamelose, idose, lyxose, mannose, ribose, tagatose, and xylose. Exemplary disaccharides can include for example cellobiose, gentiobiose, lactose, lactulose, maltose, melibiose, primeverose, rutinose, scillabiose, sophorose, turanose, and vicianose. In embodiments, sucrose, trehalose, fructose, maltose, or combinations thereof can be utilized. All optical isomers of exemplified sugars (D, L, and racemic mixtures) are also included herein. Exemplary sugar alcohols can include sorbitol, mannitol, xylitol, erythritol, ribitol, and inositol.

In some embodiments, the compositions can include at least one thickener. Suitable thickeners can include for example hydroxyethyl cellulose (HEC), methyl cellulose (MC), microcrystalline cellulose, hydroxypropyl methyl cellulose (HPMC), hydroxyethylmethyl cellulose (HEMC), hydroxypropyl cellulose (HPC), dextran, polyvinylpyrrolidone, and mixtures thereof.

In embodiments, disclosed compositions or formulations can include at least one buffer. A buffer can generally function to stabilize the pH of the composition. The particular buffer to be utilized can depend at least in part on the particular vaccine (or vaccines) that are included in the composition. The pH of the composition can be important, for example, to maintain the solubility of the vaccine at a desired level. Generally, any commonly utilized buffers can be used in disclosed compositions.

Exemplary buffers can include for example, histidine, phosphate buffers, acetate buffers, citrate buffers, glycine buffers, ammonium acetate buffers, succinate buffers, pyrophosphate buffers, Tris acetate (TA) buffers, and Tris buffers. Buffered saline solutions can also be utilized as buffers. Exemplary buffered saline solutions include, for example, phosphate buffered saline (PBS), Tris buffered saline (TBS), saline-sodium acetate buffer (SSA), saline-sodium citrate buffer (SSC). In embodiments, PBS can be utilized as the buffer.

In some embodiments, the buffer may be used to replace some or all of the water present in the aluminum-containing wet gel suspension. This can be accomplished by, e.g., serially centrifuging the aluminum-containing wet gel suspension, removing supernatant, and adding buffer until the desired amount of water has been replaced by buffer. The desired amount of buffer and/or water will depend on the vaccine (or vaccines) used, excipients used, desired coating properties, and desired amount of aluminum present in the final compositions. In some embodiments, the compositions can include one or more additional excipients. An excipient can function to maintain the active nature of the vaccine, to facilitate the coating performance of the formulation, or a combination thereof. The particular excipient to be utilized can depend at least in part on the particular vaccine (or vaccines) that are included in the formulation.

Exemplary optional additional excipients can include for example co-adjuvants, carbohydrates, polymers, amino acids, polyamino acids, surfactants, proteins, non-aqueous solvents, inorganic salts, acids, bases, antioxidants and saccharin.

Compositions can also include additional components, such as a second (or subsequent) vaccine or other active pharmaceutical ingredient (API), a second (or subsequent) sugar (or sugar alcohol, or combinations thereof), thickener, buffer, or other excipient, components not noted herein, or some combination thereof.

The amounts of the various components in disclosed compositions can vary depending on the identity of the components in the aqueous formulation, the amount of vaccine and/or aluminum desired on the microneedle array, the type of microneedle array being coated, other considerations not discussed herein, or some combination thereof. In some embodiments, disclosed compositions can have an overall solids content from 10% to 70% by weight; from 20% to 60% by weight; or from 30% to 55% by weight.

Compositions can also be characterized based on the amount of vaccine in the formulation. In some embodiments, a disclosed formulation can have from 0.01% to 80% by weight of the at least one vaccine; 0.5% to 70% by weight of the at least one vaccine; or from 0.5% to 50% by weight of the at least one vaccine.

Compositions can also be characterized based on the amount of sugar (in some embodiments, sugar alcohol, or combinations of sugars, sugar alcohols, or both sugar(s) and sugar alcohol(s)) in the formulation. In some embodiments, a disclosed formulation can have from 0% to 60% by weight of at least one sugar, sugar alcohol, or combinations thereof; or from 5% to 60% by weight of at least one sugar, sugar alcohol, or combinations thereof.

Compositions can also be characterized based on the amount of thickener in the formulation. In some embodiments, a disclosed formulation can have from 0% to 60% by weight of at least one thickener; or from 5% to 60% by weight of at least one thickener. Thickeners, if utilized, can be used to increase the viscosity of the formulation.

Compositions can also be characterized based on the amount of aluminum in the formulation. In some embodiments, a disclosed formulation can have from 0.01% to 10% by weight of aluminum; from 0.01% to 5% by weight of aluminum, from 1% to 5% by weight of aluminum, from 3% to 5% by weight of aluminum, from 0.01% to 3% by weight of aluminum, from 0.5% to 2.5% by weight of aluminum, or from 1% to 2% by weight of aluminum.

Compositions can also be characterized based on the amount of aluminum-containing wet gel suspension added to the excipients to make the composition. In some embodiments, a disclosed composition can comprise from 10% to 70% by weight aluminum-containing wet gel suspension; or from 40% to 60% by weight aluminum-containing wet gel suspension. In some embodiments, a disclosed composition can comprise 50% by weight aluminum-containing wet gel suspension.

Compositions can also be characterized based on the amount of buffer in the formulation. In some embodiments, a disclosed formulation can have from 1% to 20% by weight of buffer.

In some embodiments, a composition described herein can be further characterized by its viscosity. Generally, viscosity is a measurement of the resistance of a fluid which is being deformed by either shear stress or tensile stress. In some embodiments, disclosed compositions can be characterized by their resistance to being deformed by a shear stress, which can also be referred to as the shear viscosity of the formulation. Various instruments can be used for viscosity testing, including rheometers. In some embodiments, the viscosity of a formulation can be measured using a rheometer, for example rheometers from TA Instruments (New Castle, Del.).

Generally, if a composition is too viscous, the formulation will be difficult to utilize in manufacturing methods, can produce non-reproducible coatings (and therefore non-reproducible amounts of vaccine and alum that will be administered by the microneedle array upon use) and can result in an overall reduction in the coating weight. If a composition is not viscous enough, the formulation will not be able to effectively coat the microneedle surfaces (which could therefore require more dips of the microneedle in the formulation, thereby increasing the manufacturing costs) and in some cases capillary forces can cause the formulation to coat the microneedle and the microneedle substrate, which is sometimes referred to as “capillary jump”. The desired viscosity of a composition can depend at least in part on the geometry of the microneedles, the particular coating method being utilized, the desired number of coating steps, other considerations not discussed herein, or some combination thereof.

In some embodiments, compositions disclosed herein can have a viscosity (or shear viscosity) of from 500 to 30,000 centipoise (cps) when measured at a shear rate of 100 s⁻¹ at a temperature of 25° C. In embodiments, compositions disclosed herein can have a viscosity (or shear viscosity) of from 500 to 10,000 cps when measured at a shear rate of 100 s⁻¹ at a temperature of 25° C. In embodiments, compositions disclosed herein can have a viscosity (or shear viscosity) of from 500 to 8,000 cps when measured at a shear rate of 100 s⁻¹ at a temperature of 25° C.

In some embodiments, the compositions are uniformly suspended, or can remain uniformly suspended for at least 8 hours, at least 10 hours, or more. By uniformly suspended, it is meant that the compositions are stable and resistant to settling when not agitated for at least 8 hours, at least 10 hours, or more. The nature of the compositions and their uniform stability allows simpler coating of microneedles or microneedle arrays with maximal amount of vaccine, adjuvanted vaccine, and/or aluminum using fewer coats.

Also disclosed herein are microneedle devices. In some embodiments, the devices comprise a microneedle array. Generally, a microneedle array can include a substrate and a plurality of microneedles positioned on the substrate.

Microneedle arrays useful for practicing the present disclosure can have a variety of configurations and features, such as those described in the following patents and patent applications, the disclosures of which are incorporated herein by reference. One embodiment for the microneedle arrays includes the structures disclosed in U.S. Patent Application Publication No. 2005/0261631 (Clarke et al.), which describes microneedles having a truncated tapered shape and a controlled aspect ratio. Another embodiment for the microneedle arrays includes the structures disclosed in U.S. Pat. No. 6,091,975 (Daddona et al.), which describes blade-like microprotrusions for piercing the skin. Still another embodiment for the microneedle arrays includes the structures disclosed in U.S. Pat. No. 6,312,612 (Sherman et al.), which describes tapered structures having a hollow central channel. Yet still another embodiment for the microneedle arrays includes the structures disclosed in U.S. Pat. No. 6,379,324 (Gartstein et al.), which describes hollow microneedles having at least one longitudinal blade at the top surface of the tip of the microneedle. A further embodiment for the microneedle arrays includes the structures disclosed in U.S. Patent Application Publication Nos. US2012/0123387 (Gonzalez et al.) and US2011/0213335 (Burton et al.), which both describe hollow microneedles. A still further embodiment for the microneedle arrays includes the structures disclosed in U.S. Pat. No. 6,558,361 (Yeshurun) and U.S. Pat. No. 7,648,484 (Yeshurun et al.), which both describe hollow microneedle arrays and methods of manufacturing thereof.

Various embodiments of microneedles that can be employed in the microneedle arrays of the present disclosure are described in PCT Publication No. WO 2012/074576 (Duan et al.), which describes liquid crystalline polymer (LCP) microneedles; and PCT Publication No. WO 2012/122162 (Zhang et al.), which describes a variety of different types and compositions of microneedles that can be employed in the microneedles of the present disclosure.

In some embodiments, the microneedle material can be (or include) silicon, glass, or a metal such as stainless steel, titanium, or nickel titanium alloy. In some embodiments, the microneedle material can be (or include) a polymeric material, preferably a medical grade polymeric material. Exemplary types of medical grade polymeric materials include polycarbonate, liquid crystalline polymer (LCP), polyether ether ketone (PEEK), cyclic olefin copolymer (COC), polybutylene terephthalate (PBT). Preferred types of medical grade polymeric materials include polycarbonate and LCP.

In some embodiments, the microneedle material can be (or include) a biodegradable polymeric material, preferably a medical grade biodegradable polymeric material. Exemplary types of medical grade biodegradable materials include polylactic acid (PLA), polyglycolic acid (PGA), PGA and PLA copolymer, polyester-amide polymer (PEA).

In some embodiments, the microneedles can be a prepared from a dissolvable, degradable, or disintegradable material referred to herein as “dissolvable microneedles”. A dissolvable, degradable, or disintegradable material is any solid material that dissolves, degrades, or disintegrates during use. In particular, a “dissolvable microneedle” dissolves, degrades, or disintegrates sufficiently in the tissue underlying the stratum corneum to allow a therapeutic agent to be released into the tissue. The therapeutic agent may be coated on or incorporated into a dissolvable microneedle. In some embodiments, the dissolvable material is selected from a carbohydrate or a sugar. In some embodiments, the dissolvable material is polyvinyl pyrrolidone (PVP). In some embodiments, the dissolvable material is selected from the group consisting of hyaluronic acid, carboxymethylcellulose, hydroxypropylmethylcellulose, methylcellulose, polyvinyl alcohol, sucrose, glucose, dextran, trehalose, maltodextrin, and a combination thereof.

In some embodiments, the microneedles can be made from (or include) a combination of two or more of any of the above mentioned materials. For example, the tip of a microneedle may be a dissolvable material, while the remainder of the microneedle is a medical grade polymeric material.

A microneedle or the plurality of microneedles in a microneedle array useful for practicing the present disclosure can have a variety of shapes that are capable of piercing the stratum corneum. In some embodiments, one or more of the plurality of microneedles can have a square pyramidal shape, triangular pyramidal shape, stepped pyramidal shape, conical shape, microblade shape, or the shape of a hypodermic needle. In some embodiments, one or more of the plurality of microneedles can have a square pyramidal shape. In some embodiments, one or more of the plurality of microneedles can have a triangular pyramidal shape. In some embodiments, one or more of the plurality of microneedles can have a stepped pyramidal shape. In some embodiments, one or more of the plurality of microneedles can have a conical shape. In some embodiments, one or more of the plurality of microneedles can have a microblade shape. In some embodiments, one or more of the plurality of microneedles can have the shape of a hypodermic needle. The shape can be symmetric or asymmetric. The shape can be truncated (for example, the plurality of microneedles can have a truncated pyramid shape or truncated cone shape). In a preferred embodiment, the plurality of microneedles in a microneedle array each have a square pyramidal shape.

In some embodiments, the plurality of microneedles in a microneedle array are solid microneedles (that is, the microneedles are solid throughout). In a preferred embodiment, the plurality of microneedles in a microneedle array are solid microneedles. In some embodiments, the plurality of solid microneedles in a microneedle array can have a square pyramidal shape, triangular pyramidal shape, stepped pyramidal shape, conical shape, or microblade shape. In a preferred embodiment, the plurality of solid microneedles in a microneedle array each have a square pyramidal shape.

In some embodiments, the plurality of microneedles in a microneedle array are hollow microneedles (that is, the microneedles contain a hollow bore through the microneedle). The hollow bore can be from the base of the microneedle to the tip of the microneedle or the bore can be from the base of the microneedle to a position offset from the tip of the microneedle. In some embodiments, one or more of the plurality of hollow microneedles in a microneedle array can have a conical shape, cylindrical shape, square pyramidal shape, triangular pyramidal shape, or the shape of a hypodermic needle.

In some embodiments, each of the plurality of microneedles (or the average of all of the plurality of microneedles) has a height of less than about 1500 micrometers. In other embodiments, each of the plurality of microneedles (or the average of all of the plurality of microneedles) has a height of less than about 1200 micrometers. In still other embodiments, each of the plurality of microneedles (or the average of all of the plurality of microneedles) has a height of less than about 1200 micrometers. In yet still other embodiments, each of the plurality of microneedles (or the average of all of the plurality of microneedles) has a height of less than about 1000 micrometers. In further embodiments, each of the plurality of microneedles (or the average of all of the plurality of microneedles) has a height of less than about 750 micrometers. In still further embodiments, each of the plurality of microneedles (or the average of all of the plurality of microneedles) has a height of less than about 600 micrometers.

In some embodiments, each of the plurality of microneedles (or the average of all of the plurality of microneedles) has a height of at least about 100 micrometers. In other embodiments, each of the plurality of microneedles (or the average of all of the plurality of microneedles) has a height of at least about 200 micrometers. In still other embodiments, each of the plurality of microneedles (or the average of all of the plurality of microneedles) has a height of at least about 250 micrometers. In further embodiments, each of the plurality of microneedles (or the average of all of the plurality of microneedles) has a height of at least about 500 micrometers. In still further embodiments, each of the plurality of microneedles (or the average of all of the plurality of microneedles) has a height of at least about 800 micrometers.

In some embodiments, each of the plurality of microneedles (or the average of all of the plurality of microneedles) has a height of about 100 to about 1500 micrometers, about 200 to about 1200 micrometers, about 200 to about 1000 micrometers, about 200 to about 750 micrometers, about 200 to about 600 micrometers, or about 500 micrometers.

A single microneedle or the plurality of microneedles in a microneedle array can also be characterized by their aspect ratio. The aspect ratio of a microneedle is the ratio of the height of the microneedle, h to the width (at the base of the microneedle), w. The aspect ratio can be presented as h:w. In some embodiments, each of the plurality of microneedles (or the average of all the plurality of microneedles) has (have) an aspect ratio in the range of 2:1 to 5:1. In some of these embodiments, each of the plurality of microneedles (or the average of all of the plurality of microneedles) has (have) an aspect ratio of at least 3:1.

In some embodiments, the array of microneedles contains about 100 to about 1200 microneedles per cm² of the array of microneedles.

In some embodiments, the array of microneedles contains about 200 to about 500 microneedles per cm² of the array of microneedles.

In some embodiments, the array of microneedles contains about 300 to about 400 microneedles per cm² of the array of microneedles.

In some embodiments, each of the plurality of microneedles (or the average of all of the plurality of microneedles) in a microneedle array can penetrate into the skin to a depth of about 50 to about 1200 micrometers, about 50 to about 400 micrometers, or about 50 to about 250 micrometers.

In some embodiments, each of the plurality of microneedles (or the average of all of the plurality of microneedles) in a microneedle array can penetrate into the skin to a depth of about 100 to about 400 micrometers, or about 100 to about 300 micrometers.

In some embodiments, each of the plurality of microneedles (or the average of all of the plurality of microneedles) in a microneedle array can penetrate into the skin to a depth of about 120 to about 1200 micrometers, or about 800 to about 1200 micrometers.

In some embodiments, each of the plurality of microneedles (or the average of all of the plurality of microneedles) in a microneedle array can penetrate into the skin to a depth of about 400 to about 800 micrometers.

For all of the above embodiments, it will be appreciated that the depth of penetration (DOP) of each of the plurality of microneedles (or the average of all of the plurality of microneedles) in a microneedle array may not be the full length of the microneedles themselves.

In some embodiments of microneedle arrays, the average spacing between adjacent microneedles (as measured from microneedle tip to microneedle tip) is between about 200 micrometers and about 2000 micrometers. In other embodiments of microneedle arrays, the average spacing between adjacent microneedles is between about 200 micrometers and about 600 micrometers. In still other embodiments of microneedle arrays, the average spacing between adjacent microneedles is between about 200 micrometers and about 300 micrometers. In yet still other embodiments of microneedle arrays, the average spacing between adjacent microneedles is between about 500 micrometers and about 600 micrometers.

In some embodiments of microneedle arrays, the average spacing between adjacent microneedles (as measured from microneedle tip to microneedle tip) is greater than about 200 micrometers. In other embodiments of microneedle arrays, the average spacing between adjacent microneedles is greater than about 500 micrometers.

In some embodiments of microneedle arrays, the average spacing between adjacent microneedles is less than about 2000 micrometers. In other embodiments of microneedle arrays, the average spacing between adjacent microneedles is less than about 1000 micrometers. In still other embodiments of microneedle arrays, the average spacing between adjacent microneedles is less than about 600 micrometers. In yet still other embodiments of microneedle arrays, the average spacing between adjacent microneedles is less than about 300 micrometers.

The microneedle arrays can be manufactured in any suitable way such as by injection molding, compression molding, metal injection molding, stamping, photolithography, or extrusion.

The surface of the microneedles may be altered with a surface pre-treatment, such as a plasma treatment capable of altering surface functionality. For example, polycarbonate may be plasma treated with a nitrogen plasma to cause amide functionalization or with an oxygen plasma to cause carboxylate functionalization. A combination of nitrogen and oxygen plasma treatment may be used to give a mixed surface functionality. Alternatively, the surface of the microneedles may be treated with a coating to alter the surface properties. Such a coating may be directly applied as a solid material, such as through use of heat or plasma deposition. Examples of thin layers of material cured onto the array include plasma deposited diamond-like glass films, such as those described in United States Patent No. 6,881,538 (the disclosure of which is incorporated herein by reference thereto), ultraviolet polymerized acrylates, such as those described in U.S. Pat. No. 5,440,446 (the disclosure of which is incorporated herein by reference thereto), plasma deposited fluoropolymers, or any other thin layer that may be applied by conventional coating method, such as spray coating or roll coating and subsequently crosslinked using any suitable radiation. In one embodiment, a diamond-like glass film may be deposited on the microneedles and subsequently treated with an oxygen plasma to make the surface hydrophilic.

The compositions and formulations of the present invention of the present invention can be coated on microneedle devices, arrays and microneedles.

As described above, the coating compositions generally comprise an aluminum-containing wet gel suspension (in some embodiments, a concentrated aluminum-containing wet gel suspension), and a vaccine. In some embodiments, the coating compositions further comprise a sugar, sugar alcohol, or combinations thereof. In some embodiments, the compositions further comprise a thickener. In some embodiments, the compositions comprise a buffer. In some embodiments, the buffer is part of the aluminum-containing wet gel suspension. In some embodiments, the compositions further comprise additional optional excipients. The amount of the coating composition applied to the microneedles may be adjusted depending on the desired application.

Generally, the water present in the composition (in some embodiments, the water present is part of the aluminum-containing wet gel suspension and/or buffer) is evaporated after application to the microneedle array to leave dried coating material on the microneedle array. Evaporation may be allowed to take place at ambient conditions or may be adjusted by altering the temperature or pressure of the atmosphere surrounding the microneedle array. Evaporation conditions are desirably selected so as to avoid degradation of the coating material.

Dried coating material is deposited on the microneedle array upon evaporation of the transferred coating solution. In one embodiment, the dried coating material is preferentially deposited on the microneedles. By preferentially deposited it is meant that the amount of dried coating per unit surface area will be greater on the microneedles than on the substrate. More preferably, the dried coating material is preferentially deposited on or near the tips of the microneedles. In some cases more than half of the dried coating material by weight is deposited on the microneedles. In some cases the dried coating preferentially resides on the upper half of the microneedles, that is, the portion of the microneedles away from the substrate. In one embodiment substantially no dried coating material is deposited on the substrate, that is, substantially all of the dried coating material is deposited on the microneedles. In one embodiment, substantially all of the dried coating material is deposited on the upper half of the microneedles. The thickness of the dried coating material may vary depending on the location on the microneedle array and the intended application use for the coated microneedle array. Typical dried coating thicknesses are less than 50 microns, often less than 20 microns and sometimes less than 10 microns. It may be desirable for the coating thickness to be smaller near the tip of the microneedle so as not to interfere with the ability of the microneedle to effectively pierce into the skin.

In one embodiment, the dried coating material contains a vaccine and the vaccine is preferentially deposited on the microneedles. By preferentially deposited it is meant that the amount of vaccine per unit surface area will be greater on the microneedles than on the substrate. More preferably, the vaccine is preferentially deposited on or near the tips of the microneedles. In some cases more than half of the vaccine by weight is deposited on the microneedles. In some cases the vaccine preferentially resides on the upper half of the microneedles, that is, the portion of the microneedles away from the substrate. In one embodiment substantially no vaccine is deposited on the substrate, that is, substantially all of the vaccine is deposited on the microneedles. In one embodiment, substantially all of the vaccine is deposited on the upper half of the microneedles.

In one embodiment, the dried coating material contains aluminum (in some embodiments, the aluminum is in the form on an aluminum salt, such as aluminum hydroxide or aluminum phosphate; in some embodiments, the aluminum is adjuvanted to a vaccine) and the aluminum is preferentially deposited on the microneedles. By preferentially deposited it is meant that the amount of aluminum per unit surface area will be greater on the microneedles than on the substrate. More preferably, the aluminum is preferentially deposited on or near the tips of the microneedles. In some cases more than half of the aluminum by weight is deposited on the microneedles. In some cases the aluminum preferentially resides on the upper half of the microneedles, that is, the portion of the microneedles away from the substrate. In one embodiment substantially no aluminum is deposited on the substrate, that is, substantially all of the aluminum is deposited on the microneedles. In one embodiment, substantially all of the aluminum is deposited on the upper half of the microneedles.

In one embodiment, the microneedle arrays described herein may be applied to a skin surface in the form of a patch, such as, e.g., a patch comprising an array, pressure sensitive adhesive, and backing. The microneedles of the array may be arranged in any desired pattern or distributed over the microneedle substrate surface randomly. In one embodiment, arrays of the present invention have a distal-facing surface area of more than about 0.1 cm² and less than about 20 cm², preferably more than about 0.5 cm² and less than about 5 cm². In one embodiment, a portion of the substrate surface of the patch is non-patterned. In one embodiment the non-patterned surface has an area of more than about 1 percent and less than about 75 percent of the total area of the device surface that faces a skin surface of a patient. In one embodiment the non-patterned surface has an area of more than about 0.10 square inch (0.65 cm²) to less than about 1 square inch (6.5 cm²). In another embodiment, the microneedles are disposed over substantially the entire surface area of the array.

FIG. 2 shows a photomicrograph of a portion of a microneedle array 20, having a plurality of microneedles 21. The microneedles 21 are coated with a coating 22 formed from one embodiment of the compositions described herein (the coating of Example 3). Each microneedle 21 may have a height h, which is the length from the tip 23 of the microneedle to the bottom 24 of the microneedle at the microneedle substrate 25. Either the height of a single microneedle or the average height of all microneedles on the microneedle array can be referred to as the height of the microneedle, h. In embodiments, each of the plurality of microneedles (or the average of all of the plurality of microneedles) can have a height of about 1 to 1200 micrometers (μm). In embodiments, each of the plurality of microneedles can have a height of about 1 to 1000 In embodiments, each of the plurality of microneedles can have a height of about 200 to 750

In FIG. 2, the coated material has formed a “teardrop” shape near the tip 23 of the microneedle 21. This shape may be particularly desirable as it concentrates material near the tip of the microneedle, but does not appreciably alter the tip geometry, thus allowing for efficient piercing of the skin and delivery of coated material into the skin. The teardrop shape may be generally characterized by the maximum dimension of the dried coating when observed from above (i.e., looking down at the shaft of the needle 21 towards the microneedle array substrate 25) and the height above the substrate 25 where the maximum dimension of the dried coating occurs.

In some embodiments, the coated microneedle devices have a surface area, In some embodiments, the coated microneedle devices comprise at least 0.03 micrograms of aluminum per cm\hat{ }2 surface area of a microneedle array; at least 1 microgram of aluminum per cm\hat{ }2 surface area of a microneedle array; at least 3 micrograms of aluminum per cm\hat{ }2 surface area of a microneedle array, at least 8 micrograms of aluminum per cm\hat{ }2 surface area of a microneedle array, at least 10 micrograms of aluminum per cm\hat{ }2 surface area of a microneedle array, at least 12 micrograms of aluminum per cm\hat{ }2 surface area of a microneedle array, or at least 15 micrograms of aluminum per cm\hat{ }2 surface area of a microneedle array. In some embodiments, the coated microneedle devices comprise from 0.03 to 18 micrograms of aluminum per cm\hat{ }2 surface area of a microneedle array; from 3 to 15 micrograms of aluminum per cm\hat{ }2 surface area of a microneedle array; or from 6 to 12 micrograms of aluminum per cm\hat{ }2 surface area of a microneedle array.

In some embodiments, the coated microneedle devices comprise at least 0.03 micrograms of aluminum per microneedle array; at least 1 microgram of aluminum per microneedle array; at least 3 micrograms of aluminum per microneedle array, at least 8 micrograms of aluminum per microneedle array, at least 10 micrograms of aluminum per microneedle array, at least 12 micrograms of aluminum per microneedle array, or at least 15 micrograms of aluminum per microneedle array. In some embodiments, the coated microneedle devices comprise from 0.03 to 18 micrograms of aluminum per microneedle array; from 3 to 15 micrograms of aluminum per microneedle array; or from 6 to 12 micrograms of aluminum per microneedle array.

Also disclosed herein are methods of forming a coated microneedle array. Such methods generally include a step of providing a microneedle array. The step of providing the microneedle array can be accomplished by manufacturing the microneedle array, obtaining a microneedle array (for example by purchasing the microneedle array), or by some combination thereof.

Methods of coating microneedle arrays can be used to form coated microneedle arrays. A coated microneedle array can include a plurality of microneedles and a coating composition on at least a portion of the plurality of microneedles.

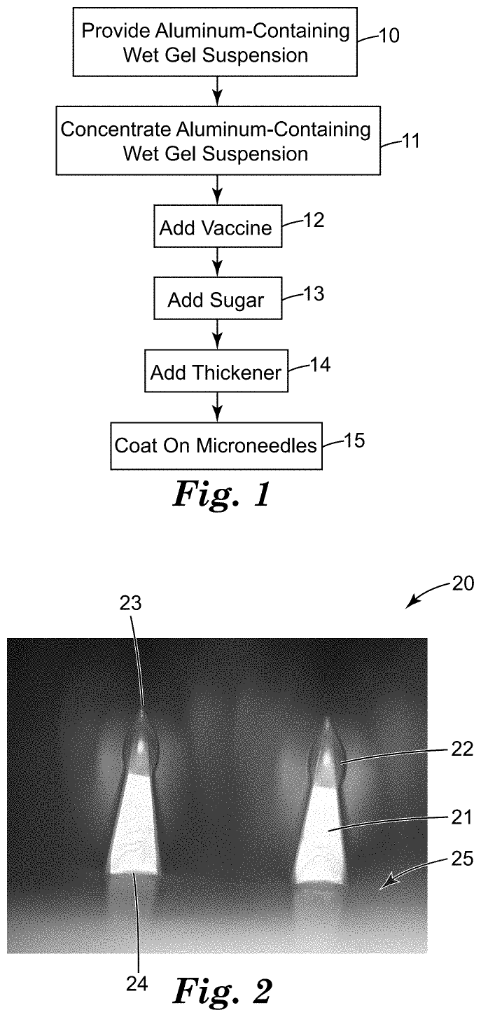

Also disclosed herein are methods of forming an alum-adjuvanted vaccine formulation. One embodiment of the method of forming the alum-adjuvanted vaccine formulation of the present invention is shown in the flowchart of FIG. 1. Generally, such methods include providing 10 a first aluminum-containing wet gel suspension selected from aluminum hydroxide wet gel suspension and aluminum phosphate wet gel suspension, concentrating 11 the aluminum-containing wet gel suspension to produce a second, concentrated aluminum-containing wet gel suspension, and adding and mixing 12 at least one vaccine into the second aluminum-containing wet gel suspension in an amount effective to stimulate an immune response in a mammal to form the alum-adjuvanted vaccine formulation. In some embodiments, the method further comprises the optional step(s) of adding a sugar (in some embodiments, a sugar alcohol, combinations of sugars, combinations of sugar alcohols, or combinations of sugar(s) and sugar alcohol(s)) 13, and a thickener 14 and mixing the sugar or sugar alcohol and thickener into the alum-adjuvanted vaccine formulation. Other optional excipients, such as those described above may be added as well (not shown). In some embodiments, the other optional excipients may be added just before, during or just after the step of adding the sugar or sugar alcohol. In some embodiments, all other optional excipients are added before adding the thickener. In some embodiments, one optional excipient, a buffer, may be added before, during, or after the step of adding and mixing the sugar into the formulation. As described elsewhere herein, the buffer may also be added during the step of concentrating the aluminum-containing wet gel suspension. The sugars or sugar alcohols, thickeners, buffers and other optional excipients are described above. Once the formulation is formed, it can be 15 coated onto microneedles, stored for later coating or distribution, or distributed to coating sites. In some embodiments, the steps of adding the sugar or sugar alcohol, thickener, buffer, or optional other excipients can be combined into a single step (not shown), or into a series of combined steps (not shown), such as, for example, adding the sugar or sugar alcohol and optional excipients in the same step, then adding thickener in a separate step.

In general, the aluminum-containing wet gel suspensions comprise water and an aluminum salt, such as aluminum hydroxide or aluminum phosphate. The step of concentrating the aluminum-containing wet gel suspension to produce a second, concentrated aluminum-containing wet gel suspension can comprise any method of concentrating generally known in the art. For example, in some embodiments, the aluminum-containing wet gel suspension can be concentrated by evaporating some of the water from the aluminum-containing wet gel suspension. In some embodiments, the step of concentrating aluminum-containing wet gel suspension can be accomplished by centrifuging the aluminum-containing wet gel suspension to separate at least a portion of the water from the suspension (e.g., the supernatant), and then removing at least a portion of the supernatant.

In some embodiments, the first aluminum-containing wet gel suspension has a first aluminum concentration and the second aluminum-containing wet gel suspension has a second aluminum concentration, and the second aluminum concentration is at least 1.2 times greater than first aluminum concentration. In some embodiments, the second aluminum concentration is from 1.2 to 2 times greater than first aluminum concentration. In some embodiments, the second aluminum concentration is from 1.5 to 2 times greater than first aluminum concentration. For example the first and second aluminum concentrations can be described by mg/ml. As used herein, aluminum concentration means the concentration of elemental aluminum.

In some embodiments, the first aluminum-containing wet gel suspension has a first volume and concentrating the aluminum-containing wet gel suspension to produce a second aluminum-containing wet gel suspension reduces the first volume such that the second aluminum-containing wet gel suspension has a second volume that is less than the first volume. In some embodiments, the second volume is at least 20% less than the first volume; at least 35% less than the first volume; at least 50% less than the first volume; at least 60% less than the first volume; at least 70% less than the first volume; at least 80% less than the first volume. In some embodiments, the second volume is from about 20% to about 80% less than the first volume; from about 20% to about 70% less than the first volume; from about 30% to about 60% less than the first volume. In some embodiments, the second volume is about 50% less than the first volume.

In general, the step of mixing at least one vaccine into the second aluminum-containing wet gel suspension includes any method of mixing known in the art, such as, for example, placing the vaccine into the suspension and manually mixing the vaccine into the suspension. In some embodiments, mixing includes vortexing, vibrating, swirling, or otherwise agitating the suspension once the vaccine has been placed into it. In some embodiments, the mixture of the aluminum-containing wet gel suspension and the at least one vaccine may be allowed to rest for a desired period of time, such as 1 hour, 2 hours, 1 to 8 hours, 1 to 10 hours, or more. Such rest time will depend on the type of vaccine used and the desired application.

In general, the step of mixing at least one vaccine into the second aluminum-containing wet gel suspension occurs after concentrating the aluminum-containing wet gel suspension and prior to mixing in any sugar, sugar alcohol, thickener, or other excipients used. In some embodiments, the buffer may be mixed into the first or second aluminum-containing wet gel suspension or used to replace the water of the aluminum-containing wet gel suspension prior to addition of the vaccine.

In general the step or steps of mixing a sugar or sugar alcohol, thickener, buffer, or combinations thereof into the alum-adjuvanted vaccine formulation comprises the same methods described above for mixing the vaccine into the aluminum-containing wet gel suspension. In some embodiments, the sugar or sugar alcohol, thickener, buffer, or combinations thereof are mixed into the alum-adjuvanted vaccine formulation until the sugar or sugar alcohol, thickener, buffer, or combinations thereof are fully dissolved. In some embodiments, the sugar or sugar alcohol, thickener, buffer, or combinations thereof are mixed into the alum-adjuvanted vaccine formulation until the sugar or sugar alcohol, thickener, buffer, or combinations thereof are partially dissolved.

Also disclosed herein are methods for maximizing the alum content of a vaccine-coated microneedle array and methods for forming a vaccine and adjuvant coated microneedle array. In general, the methods comprise providing a microneedle array comprising a microneedle substrate and a plurality of microneedles, forming alum-adjuvanted vaccine formulation according to the methods described herein, and bringing at least a portion of the plurality of microneedles into contact with the alum-adjuvanted vaccine formulation, thereby transferring at least a portion of the alum-adjuvanted vaccine formulation to the microneedle array to form a wet-coated microneedle array.

The step of bringing at least a portion of the plurality of microneedles into contact with the alum-adjuvanted vaccine formulation can comprise any microneedle coating methods known in the art. For example, the formulations can be applied to the microneedles by dip-coating such as described, for example, in U.S. Pat. No. 8,414,959 (Choi et al.), U.S. Patent Application Publication No. 2014/006842 (Zhang et al.), and U.S. Patent Application Publication No. 2013/0123707 (Determan et al.), the disclosures of which are incorporated herein by reference.

The step of contacting the microneedles with the formulation can be carried out more than once. For example, once the contact between the microneedles and the formulation has been terminated, the microneedles and the formulation can be brought into contact again. The optional second (and optional subsequent) steps of contacting the microneedles and the formulation can be carried out immediately, or there can be a delay between the contact steps.

The methods can additionally comprise drying the wet-coated microneedle array to form a coated microneedle array. Drying methods that can be utilized, such as, for example, evaporating, are described above.

Also disclosed herein are methods of delivering an alum-adjuvanted vaccine to a mammal comprising providing a microneedle array comprising a microneedle substrate and a plurality of microneedles, forming alum-adjuvanted vaccine formulation as described herein, bringing at least a portion of the plurality of microneedles into contact with the alum-adjuvanted vaccine formulation, thereby transferring at least a portion of the alum-adjuvanted vaccine formulation to the microneedle array to form a wet-coated microneedle array, drying the wet-coated microneedle array to form a coated microneedle array, contacting at least a portion of the mammal's skin with at least a portion of the microneedle array, and applying sufficient pressure to the microneedle array to cause the plurality of microneedles to penetrate the mammal's skin a sufficient depth for delivering the alum-adjuvanted vaccine to the mammal.

Microneedle devices may be used for immediate delivery, for example, application and immediate removal of the device from the application site, or they may be left in place for an extended time, which may range from a few minutes to as long as 1 week. In one aspect, an extended time of delivery may be from 1 to 30 minutes to allow for more complete delivery of a drug than can be obtained upon application and immediate removal. In another aspect, an extended time of delivery may be from 4 hours to 1 week to provide for a sustained release of drug.

### Embodiments

Embodiment 1 is a composition comprising:
an aluminum-containing wet gel suspension selected from aluminum hydroxide wet gel suspension and aluminum phosphate wet gel suspension;
a vaccine in an amount effective to stimulate an immune response in a mammal;
a sugar, sugar alcohol, or combinations thereof; and
a thickener;
wherein the composition has a viscosity of 500 to 30,000 cps when measured at 100 s⁻and temperature of 25° C.

Embodiment 2 is a composition according to embodiment 1, comprising a sugar, wherein the sugar is selected from raffinose, stachyose, sucrose, trehalose, apiose, arabinose, digitoxose, fucose, fructose, galactose, glucose, gulose, hamamelose, idose, lyxose, mannose, ribose, tagatose, xylose, cellobiose, gentiobiose, lactose, lactulose, maltose, melibiose, primeverose, rutinose, scillabiose, sophorose, turanose, and vicianose.

Embodiment 3 is a composition according to embodiment 2, wherein the sugar is a non-reducing sugar.

Embodiment 4 is a composition according to embodiment 3, wherein the sugar is selected from raffinose, stachyose, sucrose, and trehalose.

Embodiment 5 is a composition according to embodiment 1, comprising a sugar alcohol, wherein the sugar alcohol is selected from sorbitol, mannitol, xylitol, erythritol, ribitol, and inositol.

Embodiment 6 is a composition according to any one of the preceding embodiments, wherein the thickener is selected from hydroxyethyl cellulose, methyl cellulose, microcrystalline cellulose, hydroxypropyl methyl cellulose, hydroxyethylmethyl cellulose, hydroxypropyl cellulose, dextran, polyvinylpyrrolidone, and mixtures thereof.

Embodiment 7 is a composition according to any one of the preceding embodiments, wherein the vaccine is selected from DNA vaccine, cellular vaccine, recombinant protein vaccine, therapeutic cancer vaccine, anthrax vaccine, flu vaccine, Lyme disease vaccine, rabies vaccine, measles vaccine, mumps vaccine, chicken pox vaccine, small pox vaccine, hepatitis A vaccine, hepatitis B vaccine, hepatitis C vaccine, pertussis vaccine, rubella vaccine, diphtheria vaccine, encephalitis vaccine, Japanese encephalitis vaccine, respiratory syncytial virus vaccine, yellow fever vaccine, ebola virus vaccine, polio vaccine, herpes vaccine, human papilloma virus vaccine, rotavirus vaccine, pneumococcal vaccine, meningitis vaccine, whooping cough vaccine, tetanus vaccine, typhoid fever vaccine, cholera vaccine, tuberculosis vaccine, severe acute respiratory syndrome vaccine, HSV-1 vaccine, HSV-2 vaccine, HIV vaccine and combinations thereof.

Embodiment 8 is a composition according to any one of the preceding embodiments, wherein the vaccine is present in an amount of from 0.5 wt.-% to 50 wt.-% of the coating formulation.

Embodiment 9 is a composition according to any one of the preceding embodiments, wherein the aluminum-containing wet gel suspension is present in an amount of from 10 wt.-% to 70 wt.-% of the coating formulation.

Embodiment 10 is a composition according to any one of the preceding embodiments, wherein the sugar, sugar alcohol, or combinations thereof is present in an amount of from 0.01 wt.-% to 60 wt.-% of the coating formulation.

Embodiment 11 is a composition according to any one of the preceding embodiments, wherein the thickener is present in an amount of from 0.01 wt.-% to 60 wt.-% of the coating formulation.

Embodiment 12 is a composition according to any one of the preceding embodiments, further comprising at least one buffer.

Embodiment 13 is a composition according to embodiment 12, wherein the buffer is present in an amount of from 1 wt.-% to 20 wt.-% of the coating formulation.

Embodiment 14 is composition according to embodiment 12, wherein the at least one buffer is selected from histidine, phosphate buffers, acetate buffers, citrate buffers, glycine buffers, ammonium acetate buffers, succinate buffers, pyrophosphate buffers, Tris acetate buffers, Tris buffers, phosphate buffered saline, Tris buffered saline, saline-sodium acetate buffer, and saline-sodium citrate buffer.

Embodiment 15 is a composition according to embodiment 14, wherein the at least one buffer is phosphate buffered saline.

Embodiment 16 is a composition according to any one of the preceding embodiments, wherein the aluminum-containing wet gel suspension comprises 0.01 wt.-% to 5 wt.-% aluminum.

Embodiment 17 is a composition according to any one of the preceding embodiments, wherein the aluminum-containing wet gel suspension comprises 0.1 wt.-% to 2 wt.-% aluminum.

Embodiment 18 is a composition according to any one of the preceding embodiments, wherein the aluminum-containing wet gel suspension comprises 5 mg/ml to 22 mg/ml aluminum.

Embodiment 19 is a composition according to any one of the preceding embodiments, comprising 0.01% to 10% by weight of aluminum.

Embodiment 20 is a composition according to any one of the preceding embodiments, comprising 0.5% to 3% by weight of aluminum.

Embodiment 21 is a composition consisting essentially of:

an aluminum-containing wet gel suspension selected from aluminum hydroxide wet gel suspension and aluminum phosphate wet gel suspension;

a vaccine in an amount effective to stimulate an immune response in a mammal;

a sugar, sugar alcohol, or combinations thereof; and

a thickener;
wherein the composition has a viscosity of 500 to 30,000 cps when measured at 1005¹ and temperature of 25° C.

Embodiment 22 is a device comprising:

a microneedle array comprising a substrate and a plurality of microneedles; and

the composition of any one of claims 1-19 coated on at least a portion of one or more of the microneedles.

Embodiment 23 is a device according to embodiment 22, wherein the device has a surface area and comprises at least 0.03 micrograms of aluminum per cm\hat{ }2 of the surface area.

Embodiment 24 is a device according to embodiment 22, wherein the device has a surface area and comprises from 0.03 to 18 micrograms of aluminum per cm\hat{ }2 of the surface area.

Embodiment 25 is a method of forming an aluminum-adjuvanted vaccine formulation comprising:
providing a first aluminum-containing wet gel suspension selected from aluminum hydroxide wet gel suspension and aluminum phosphate wet gel suspension;
concentrating the aluminum-containing wet gel suspension to produce a second aluminum-containing wet gel suspension;
mixing at least one vaccine into the second aluminum-containing wet gel suspension in an amount effective to stimulate an immune response in a mammal to form the aluminum-adjuvanted vaccine formulation.

Embodiment 26 is a method according to embodiment 25, wherein the first aluminum-containing wet gel suspension has a first aluminum concentration and the second aluminum-containing wet gel suspension has a second aluminum concentration, and the second aluminum concentration is at least 1.2 times greater than first aluminum concentration.

Embodiment 27 is a method according to embodiment 25, wherein the first aluminum-containing wet gel suspension has a first aluminum concentration and the second aluminum-containing wet gel suspension has a second aluminum concentration, and the second aluminum concentration is from 1.2 to 2 times greater than first aluminum concentration.

Embodiment 28 is a method according to embodiment 26, wherein the first aluminum-containing wet gel suspension has a first aluminum concentration and the second aluminum-containing wet gel suspension has a second aluminum concentration, and the second aluminum concentration is from 1.5 to 2 times greater than first aluminum concentration.

Embodiment 29 is a method according to embodiment 25, wherein the first aluminum-containing wet gel suspension has a first volume and concentrating the aluminum-containing wet gel suspension to produce a second aluminum-containing wet gel suspension reduces the first volume such that the second aluminum-containing wet gel suspension has a second volume that is less than the first volume.

Embodiment 30 is a method according to embodiment 29, wherein the second volume is at least 20% less than the first volume.

Embodiment 31 is a method according to embodiment 29, wherein the second volume is from 20% to 80% less than the first volume.

Embodiment 32 is a method according to any one of embodiments 25-31, further comprising mixing at least one excipient into the aluminum-adjuvanted vaccine formulation.

Embodiment 33 is a method according to embodiment 32, wherein the at least one excipient comprises a sugar, a thickener, a buffer, or combinations thereof.

Embodiment 34 is a method for maximizing the aluminum content of a vaccine-coated microneedle array comprising:
providing a microneedle array comprising a microneedle substrate and a plurality of microneedles; forming aluminum-adjuvanted vaccine formulation by

providing a first aluminum-containing wet gel suspension selected from aluminum hydroxide wet gel suspension and aluminum phosphate wet gel suspension;

concentrating the aluminum-containing wet gel suspension to produce a second aluminum-containing wet gel suspension;

mixing at least one vaccine into the second aluminum-containing wet gel suspension in an amount effective to stimulate an immune response in a mammal to form the aluminum-adjuvanted vaccine formulation; and
bringing at least a portion of the plurality of microneedles into contact with the aluminum-adjuvanted vaccine formulation, thereby transferring at least a portion of the aluminum-adjuvanted vaccine formulation to the microneedle array to form a wet-coated microneedle array.

Embodiment 35 is a method according to embodiment 34, wherein forming aluminum-adjuvanted vaccine formulation further comprises mixing at least one excipient into the aluminum-adjuvanted vaccine formulation.

Embodiment 36 is a method according to embodiment 35, wherein the at least one excipient comprises a sugar, a thickener, a buffer, or combinations thereof.

Embodiment 37 is a method according to any one of embodiments 34-36, wherein bringing at least a portion of the plurality of microneedles into contact with the aluminum-adjuvanted vaccine formulation comprises dip-coating the microneedle array.

Embodiment 38 is a method according to any one of embodiments 34-37, further comprising drying the wet-coated microneedle array to form a coated microneedle array.

Embodiment 39 is a method according to embodiment 38, wherein drying comprises allowing at least a portion of the aluminum-adjuvanted vaccine formulation to evaporate.

Embodiments 40 is a method of delivering an alum-adjuvanted vaccine to a mammal comprising:
providing a microneedle array comprising a microneedle substrate and a plurality of microneedles;
forming alum-adjuvanted vaccine formulation by

providing an aluminum-containing wet gel suspension selected from aluminum hydroxide wet gel suspension and aluminum phosphate wet gel suspension;

concentrating the aluminum-containing wet gel suspension to produce a concentrated aluminum-containing wet gel suspension;

mixing at least one vaccine into the concentrated aluminum-containing wet gel suspension in an amount effective to stimulate an immune response in a mammal to form the alum-adjuvanted vaccine formulation; and
bringing at least a portion of the plurality of microneedles into contact with the alum-adjuvanted vaccine formulation, thereby transferring at least a portion of the alum-adjuvanted vaccine formulation to the microneedle array to form a wet-coated microneedle array;
drying the wet-coated microneedle array to form a coated microneedle array;
contacting at least a portion of the mammal's skin with at least a portion of the microneedle array; and
applying sufficient pressure to the microneedle array to cause the plurality of microneedles to penetrate the mammal's skin a sufficient depth for delivering the alum-adjuvanted vaccine to the mammal.

### EXAMPLES

### Microneedle Array Fabrication

Microneedle arrays were injection molded from Class VI medical grade liquid crystalline polymer (LCP, Vectra® MT1300, Ticona Plastics, Auburn Hills, Mich.). The arrays had a surface area of approximately 1.27 cm². Each microneedle array featured 316 four-sided pyramidal-shaped microneedles that were arranged in an octagonal pattern, with microneedle heights of about 500 microns, an aspect ratio of approximately 3:1, and a tip-to-tip distance between neighboring microneedles of about 550 microns. The arrays were attached to a 5 cm² adhesive patch with 1513 double-sided medical adhesive (3M Company, St. Paul, Minn.).

### Analytical Procedure for Ovalbumin Content

The ovalbumin content of a coated microneedle array was determined by high performance liquid chromatography (HPLC). The coating formulation was extracted from a coated array by placing a coated array into a polypropylene sample cup, adding 1 mL of extraction solution (200 mcg/mL Polysorbate-80 in phosphate-buffered-saline), snapping a cap onto the sample cup, and then rocking the sample for 30 minutes. A portion (20 □L) of the extraction solution was injected into an HPLC instrument containing a ZORBAX SB300-C8 column, 50×2.1mm, 3.5 micron particle size (Agilent Technologies, Santa Clara, Calif.) that was thermostated at 60° C. The mobile phase consisted of two eluents: eluent A was water, acetonitrile and phosphoric acid (900:100:3) and eluent B was water, acetonitrile and phosphoric acid (100:900:3). The flow rate of the mobile phase was 0.4 mL/min. Ovalbumin was eluted from the column using a 5 minute gradient from 10% eluent B to 90% eluent B.

### Analytical Procedure for Aluminum Content

The aluminum content of a coated microneedle array was determined by Inductively Coupled Plasma-Atomic Emission Spectroscopy (ICP-AES). The coating formulation was extracted from a coated array by placing a coated array into a polypropylene sample cup, adding 1 mL of extraction solution (200 mcg/mL Polysorbate80 in phosphate-buffered-saline), snapping a cap onto the sample cup, and then rocking for 30 minutes. A sample of the extraction solution (0.5 mL) was added to 10 mL of 4% nitric acid solution and mixed by inversion, prior to analysis by ICP-AES.

### Procedure for the In Vivo Immuno Study

An in vivo immuno study was performed in order to compare the immune response for vaccine delivered by coated microneedle arrays to the immune response for vaccine delivered subcutaneously by a traditional needle-and-syringe method. Male Sprague-Dawley rats (CD-IGS strain from Charles River Laboratories, nominally 400 g) were used (3 animals in the coated microneedle array group and 3 animals in the comparator group (needle-and-syringe administration)). Each animal was initially anaesthetized in a chamber using 5% isoflurane in oxygen and then placed in lateral recumbancy on a thermostatically controlled surface with the nose and mouth placed inside an anesthetic face mask for the duration of the session. Isoflurane was maintained at 1.5-3.0% during the session.

For the microneedle treatment group, the application area on the shoulder was trimmed with an Oster electric clipper (#50 blade). The trimmed area was then shaved with a Remington electric razor.

For the comparator group receiving subcutaneous (SC) administration by needle-and-syringe, the injection area on the shoulder was trimmed with an Oster electric clipper. The shaved skin was cleaned by wiping with gauze pads that had been soaked with 70% isopropyl alcohol (IPA). The IPA was allowed to evaporate for at least 30 seconds prior to dosing. Adhesive patches, that contained coated microneedle arrays, were applied at the prepared application site using a mechanical applicator as described in United States Patent Application No. US2008/0039805. After each application, the patches were maintained at the application site for 15 minutes and then removed. Patches were applied on day 0 (Dose-1), day 14 (Dose-2), and day 28 (Dose-3) of the study.

The comparator group was dosed subcutaneously at the same time points using a needle-and-syringe (0.5 mL per dose bolus injection with a 20 guage-linch Monoject needle attached to a 1 mL Luer-Loc syringe, Becton-Dickinson, Franklin Lakes, N.J.), with a formulation that contained ovalbumin (30 mcg/dose) and Alhydrogel® (160 mcg-aluminum/dose). The injectable formulation for the comparator group was prepared from EndoFit ovalbumin (pyrogen-free, InvivoGen, San Diego, Calif.), Alhydrogel® 2% (Brenntag Biosector, Denmark), Polysorbate-80 (NF grade, Spectrum Chemical, New Brunswick, N.J.), ethyl alcohol (200 proof, USP grade, Aaper, Shelbyville, Ky.) and phosphate-buffered saline (PBS, 10×, HyClone Laboratories, Logan, Utah). The injectable formulation was prepared according to the following 7-step procedure. Step-1) 1X PBS was prepared by combining 50 mL of 10× PBS with 450 mL of high purity water (Milli-Q50, Millipore, Billerica, Mass.). Step-2) Ethyl alcohol (1 mL) was added to a 15 mL vial containing Polysorbate-80 (0.1 g). The vial was capped and the sample was mixed by rocking to dissolve the Polysorbate-80. Step-3) The solution of Polysorbate-80 was transferred into 500 mL of PBS and mixed by rocking. Step-4) The PBS/Polysorbate-80 solution (50 mL) was sterile filtered into a sterile screwcap vial (using a sterile Millex-GV 0.22 micron syringe-filter (33 mm diameter filter, Millipore Merck Ltd, Tullagreen, IRL) and a sterile syringe (60 mL, Becton-Dickinson)). Step-5) A 1 mg/mL stock solution of Endofit ovalbumin was prepared by weighting 0.0014 g of ovalbumin into a 2 mL screwcap vial, adding 1.4 mL of sterile-filtered PBS/Polysorbate-80 solution, and mixing by rocking for 10 minutes. Step-6) Alhydrogel® suspension (0.4 mL) and 0.6 mL of the stock ovalbumin solution (1 mg/mL) were added to a 2 mL screwcap vial and mixed by rocking for 10 minutes. Step-7) The ovalbumin-Alhydrogel mixture was transferred to a 15 mL screwcap vial and 9 mL of the PBS/Polysorbate-80 solution was added. The vial was capped and then rocked for 45 minutes to obtain the injectable formulation of ovalbumin-Alhydrogel.

Blood samples (0.8 mL) were obtained from the animals on day 0, day 14, day 28 and day 42. On each sampling day, the blood sample was drawn prior to the next dose being administered. Blood samples were drawn from the anterior vena cava by needle-and-syringe (20 guage-1 inch Monoject needle attached to a 1 mL Luer-Loc syringe, Becton-Dickinson), and then transferred to clot tubes (2 mL Monoject tube with no additive, Covidien, Mannsfield, Mass.). After 30 minutes at room temperature, the serum tubes were centrifuged to isolate the serum from the clotted red blood cells (GLS centrifuge, GH3.7 rotor, Beckman Coulter, Schaumburg, Ill.). The serum was transferred into screw-capped BioStor vials (2 mL, National Scientific, Claremont, Calif.) and then frozen on dry ice. The serum samples were subsequently stored at −80° C. until tested by ELISA for antibody titer. ELISA kits and procedures from Alpha Diagnostics, San Antonio, Tex. (610-100-OGG) were used to determine the anti-ovalbumin IgG content in the serum samples. A SPECTRAMAXplus plate reader (Molecular Devices, Sunnyvale, Calif.) was used to quantify the color intensity in the wells of the ELISA plates.

### Example 1

A formulation for coating microneedle arrays was prepared with Alhydrogel® (aluminum hydroxide gel, 10 mg-Aluminum/mL, manufactured by Brenntag Biosector), Endofit ovalbumin (pyrogen-free, InvivoGen, San Diego, Calif.), sucrose (ACS grade, Sigma) and hydroxyethylcellulose (HEC, 100 cP, NF grade, Spectrum Chemical). Alhydrogel (1 mL) was transferred to a 2 mL microfuge tube, and the tube was centrifuged (Minispin Plus, Eppendorf, Westbury, N.Y.) at 4500 rpm for 3 minutes. Supernate (0.33 mL) was removed from the tube. Ovalbumin (45 mg) was added to the tube. The tube was capped and rocked, to mix the ovalbumin and Alhydrogel. Sucrose (185 mg) and HEC (100 mg) were added to the tube, and the tube was mixed (Turbula mixer (96 revolutions/min), Glenn Mills Inc., Clifton, N.J.) to yield a thick, uniform formulation. The mixed formulation was collected at the bottom of the tube by centrifuging at 4500 rpm for 3 minutes.

A dip-coating process at ambient room conditions (20° C., 40% relative humidity) as described in U.S. Pat. No. 8,414,959 (example 16) was used to coat the ovalbumin:Alhydrogel formulation onto the tips of microneedles. For each array, three dips were performed to coat the microneedles, pausing 1.5 seconds between each dip. The arrays were allowed to dry at ambient conditions for about 30 minutes, before being stored in a light and moisture proof foil pouch (Oliver-Tolas Healthcare Packaging, Feasterville, Pa.) at room temperature.

The mean ovalbumin content per array (n=3) and the mean aluminum content (n=3) per array are reported in Table 1.

TABLE 1
Coated Microneedle Arrays of Example 1

| Ovalbumin Content | Aluminum Content |
|---|---|
| (mcg/array) | (mcg/array) |
| 29.6 | 7.4 |

### Example 2

Coated microneedle arrays prepared as described in Example 1 were prepared and evaluated using the in vivo immuno study described above (including the needle-and-syringe comparator). After dosing the rats with the microneedle arrays, the residual amount of ovalbumin on the arrays was quantified by HPLC using the procedure described above. The residual amount of ovalbumin was subtracted from the initial ovalbumin content, in order to determine the dose of ovalbumin that was delivered. There was insufficient sample to quantify both residual ovalbumin and aluminum, so the percentage of ovalbumin delivered was used to calculate the amount of aluminum that was delivered. Serum samples were tested by ELISA according to the method described above in order to quantify the antibody titer for anti-ovalbumin IgG. Table 2a and Table 2b summarize the doses of ovalbumin and aluminum delivered by needle-and-syringe administration (comparator) and by coated microneedle array administration. The corresponding antibody titer for each sample is also reported.

TABLE 2a
Dose Delivered by Needle-and-Syringe (0.5 mL) and Subsequent
anti-Ovalbumin IgG Antibody Titer (Comparative Example)

| | SC Dose-1 | | | SC Dose-2 | | | SC Dose-3 | | |
|---|---|---|---|---|---|---|---|---|---|
| | (day 0) | | Antibody | (day 14) | | Antibody | (day 28) | | Antibody |
| | Ova | Al | Titer | Ova | Al | Titer | Ova | Al | Titer |
| Rat | (mcg) | (mcg) | (day 14) | (mcg) | (mcg) | (day 28) | (mcg) | (mcg) | (day 42) |
| 1 | 30 | 160 | 4812 | 30 | 160 | 237714 | 30 | 160 | 171288 |
| 2 | 30 | 160 | 3659 | 30 | 160 | 166227 | 30 | 160 | 147811 |
| 3 | 30 | 160 | 16604 | 30 | 160 | 308416 | 30 | 160 | 195260 |

TABLE 2b
Dose Delivered by Patches with Coated Microneedle Arrays of
Example 1 and Subsequent anti-Ovalbumin IgG Antibody Titer

| | Dose-1 | | | Dose-2 | | | Dose-3 | | Antibody |
|---|---|---|---|---|---|---|---|---|---|
| | (day 0) | | Antibody | (day 14) | | Antibody | (day 28) | | Titer |
| | Ova | Al | Titer | Ova | Al | Titer | Ova | Al | (day 42) |
| Rat | (mcg) | (mcg) | (day 14) | (mcg) | (mcg) | (day 28) | (mcg) | (mcg) | Titer |
| 1 | 18.5 | 4.6 | 15977 | 10.5 | 2.6 | 204335 | 16.2 | 4.1 | 1445360 |
| 2 | 12.0 | 3.0 | 5150 | 10.7 | 2.7 | 34202 | 11.3 | 2.8 | 170836 |
| 3 | 10.3 | 2.6 | 4699 | 15.5 | 3.9 | 42107 | 14.1 | 3.5 | 118086 |

### Example 3

A formulation for coating microneedle arrays was prepared with Alhydrogel® (aluminum hydroxide gel, 10 mg-Aluminum/mL, manufactured by Brenntag Biosector), Endofit ovalbumin (pyrogen-free, Invivogen), sucrose (Aldrich Chemical) and hydroxyethylcellulose (HEC, 100 cP, NF grade, Spectrum Chemical). Alhydrogel (1 mL) was transferred to a 2 mL microfuge tube, and the tube was centrifuged (Minispin Plus, Eppendorf) at 4500 rpm for 3 minutes. Supernate (0.33 mL) was removed from the tube. Ovalbumin (6 mg) was added to the tube. The tube was capped and rocked, to mix the ovalbumin and Alhydrogel. Sucrose (214 mg) and HEC (110 mg) were added to the tube, and the tube was mixed (Turbula mixer (96 revolutions/min), Glenn Mills Inc.) to yield a thick, uniform formulation. The mixed formulation was collected at the bottom of the tube by centrifuging at 4500 rpm for 3 minutes. A dip-coating process at ambient room conditions (20° C., 40% relative humidity) as described in U.S. Pat. No. 8,414,959, example 16) was used to coat the ovalbumin:Alhydrogel formulation onto the tips of microneedles. For each array, three dips were performed to coat the microneedles, pausing 1.5 seconds between each dip. The arrays were allowed to dry at ambient conditions for about 30 minutes, before being stored in a light and moisture proof foil pouch (Oliver-Tolas Healthcare Packaging) at room temperature.

The mean ovalbumin content per array (n=3) and the mean aluminum content (n=3) per array are reported in Table 3.

TABLE 3
Coated Microneedle Arrays of Example 3

| Ovalbumin Content | Aluminum Content |
|---|---|
| (mcg/array) | (mcg/array) |
| 2.9 | 7.14 |

### Example 4

A formulation for coating microneedle arrays was prepared with Alhydrogel® (aluminum hydroxide gel, 10 mg-Aluminum/mL, manufactured by Brenntag Biosector), ovalbumin (Sigma, St. Louis, Mo.), D-sorbitol (99+%, Aldrich Chemical) and hydroxyethylcellulose (HEC, 100 cP, NF grade, Spectrum Chemical). Alhydrogel (1 mL) was transferred to a 2 mL microfuge tube, and the tube was centrifuged (Minispin Plus, Eppendorf) at 4500 rpm for 3 minutes. Supernate (0.33 mL) was removed from the tube. Ovalbumin (45 mg) was added to the tube. The tube was capped and rocked, to mix the ovalbumin and Alhydrogel. Sorbitol (185 mg) and HEC (100 mg) were added to the tube, and the tube was mixed (Turbula mixer (96 revolutions/min), Glenn Mills Inc.) to yield a thick, uniform formulation. The mixed formulation was collected at the bottom of the tube by centrifuging at 4500 rpm for 3 minutes.

### Example 5

A formulation for coating microneedle arrays was prepared with AdjuPhos® (aluminum phosphate gel, 5 mg-Aluminum/mL, manufactured by Brenntag Biosector), ovalbumin (Sigma, St. Louis, Mo.), sucrose (Aldrich Chemical) and hydroxyethylcellulose (HEC, 100 cP, NF grade, Spectrum Chemical). AdjuPhos (1 mL) was transferred to a 2 mL microfuge tube, and the tube was centrifuged (Minispin Plus, Eppendorf) at 4500 rpm for 3 minutes. Supernate (0.40 mL) was removed from the tube. Ovalbumin (40 mg) was added to the tube. The tube was capped and rocked, to mix the ovalbumin and AdjuPhos. Sucrose (120 mg) and HEC (85 mg) were added to the tube, and the tube was mixed (Turbula mixer (96 revolutions/min), Glenn Mills Inc.) to yield a thick, uniform formulation. The mixed formulation was collected at the bottom of the tube by centrifuging at 4500 rpm for 3 minutes.

### Example 6

A formulation for coating microneedle arrays was prepared with AdjuPhos® (aluminum phosphate gel, 5 mg-Aluminum/mL, manufactured by Brenntag Biosector), ovalbumin (Sigma, St. Louis, Mo.), D-Sorbitol (99+%, Aldrich) and hydroxyethylcellulose (HEC, 100 cP, NF grade, Spectrum Chemical). AdjuPhos (1 mL) was transferred to a 2 mL microfuge tube, and the tube was centrifuged (Minispin Plus, Eppendorf) at 4500 rpm for 3 minutes. Supernate (0.40 mL) was removed from the tube. Ovalbumin (40 mg) was added to the tube. The tube was capped and rocked, to mix the ovalbumin and AdjuPhos. Sorbitol (120 mg) and HEC (85 mg) were added to the tube, and the tube was mixed (Turbula mixer (96 revolutions/min), Glenn Mills Inc.) to yield a thick, uniform formulation. The mixed formulation was collected at the bottom of the tube by centrifuging at 4500 rpm for 3 minutes.

### Example 7

A formulation for coating microneedle arrays was prepared with AdjuPhos® (aluminum phosphate gel, 5 mg-Aluminum/mL, manufactured by Brenntag Biosector), ovalbumin (Sigma, St. Louis, Mo.), xylitol (99%, Alfa Aesar, Ward Hill, Mass.) and hydroxyethylcellulose (HEC, 100 cP, NF grade, Spectrum Chemical). AdjuPhos (1 mL) was transferred to a 2 mL microfuge tube, and the tube was centrifuged (Minispin Plus) at 4500 rpm for 3 minutes. Supernate (0.50 mL) was removed from the tube. Ovalbumin (45 mg) was added to the tube. The tube was capped and rocked, to mix the ovalbumin and AdjuPhos. Xylitol (100 mg) and HEC (70 mg) were added to the tube, and the tube was mixed (Turbula mixer (96 revolutions/min), Glenn Mills Inc.) to yield a thick, uniform formulation. The mixed formulation was collected at the bottom of the tube by centrifuging at 4500 rpm for 3 minutes.

1. (canceled)

2. A device comprising an array of a plurality of microneedles, wherein each microneedle in the array comprises a coating that comprises aluminum hydroxide or aluminum phosphate;
a vaccine in an amount effective to stimulate an immune response in a mammal;
a sugar, sugar alcohol, or combinations thereof; and
a thickener;
wherein the array comprises 3 to 15 micrograms of aluminum; and
wherein the array comprises at least 0.03 micrograms of alumina per cm² of surface area of the array.

3. The device of claim 1, wherein each of the plurality of microneedles has a shaft comprising a tip and a base, wherein
the coating is disposed on the shaft of each microneedle, and wherein
more of the coating is disposed on the shaft proximal to the tip than is disposed on the shaft proximal to the base.

4. The composition of claim 2, comprising a sugar, wherein the sugar is selected from raffinose, stachyose, sucrose, trehalose, apiose, arabinose, digitoxose, fucose, fructose, galactose, glucose, gulose, hamamelose, idose, lyxose, mannose, ribose, tagatose, xylose, cellobiose, gentiobiose, lactose, lactulose, maltose, melibiose, primeverose, rutinose, scillabiose, sophorose, turanose, and vicianose.

5. The composition of claim 4, wherein the sugar is a non-reducing sugar.

6. The composition of claim 2, comprising a sugar alcohol, wherein the sugar alcohol is selected from sorbitol, mannitol, xylitol, erythritol, ribitol, and inositol.

7. The composition of claim 2, wherein the thickener is selected from hydroxyethyl cellulose, methyl cellulose, microcrystalline cellulose, hydroxypropyl methyl cellulose, hydroxyethylmethyl cellulose, hydroxypropyl cellulose, dextran, polyvinylpyrrolidone, and mixtures thereof.

8. A method of making a device of claim 1, the method comprising
concentrating an first aluminum containing wet gel suspension to produce a second aluminum containing wet get suspension;
mixing a vaccine into the second aluminum containing wet gel suspension; and
bringing at least a portion of the plurality of microneedles into contact with the second aluminum containing wet gel suspension; wherein
the aluminum comprises water and
aluminum hydroxide, aluminum phosphate, or a combination thereof; and
the first aluminum wet gel suspension has a first aluminum concentration and the second aluminum wet gel suspension has a second aluminum concentration that is at least 1.2 times greater than the first aluminum concentration; and further wherein
the method further comprises the steps of mixing a sugar, sugar alcohol, or combinations thereof into the first aluminum wet gel suspension or the second aluminum wet gel suspension; and
mixing a thickener into the first aluminum wet gel suspension or the second aluminum wet gel suspension.

9. The method of claim 8, wherein the first aluminum wet gel suspension has a first volume and the second aluminum wet gel suspension has a second volume, wherein the second volume is at least 20% less than the first volume.

10. The method of claim 9 wherein the second volume is at least 50% less than the first volume.

11. The method of claim 8, wherein the second aluminum concentration is at 1.5 to 2 times greater than the first aluminum concentration.

12. The method of claim 8, further comprising, after the step of mixing a vaccine into the second aluminum containing wet gel suspension, allowing the second wet gel suspension to rest for 1 to 10 hours.

13. The method of claim 8, wherein the sugar, sugar alcohol, or combinations thereof is mixed into the second aluminum wet gel suspension.

14. The method of claim 8, wherein thickener is mixed into the second aluminum wet gel suspension.

15. The method of claim 8, wherein the step of concentrating comprises centrifuging the first aluminum wet gel suspension and removing a supernatant.

16. The method of claim 8, wherein the step of concentrating comprises evaporation.
